(19) United States
(12) Patent Application Publication
(10) Pub. No.: US 2021/0161799 A1
(43) Pub. Date: Jun. 3, 2021

(54) THERAPEUTIC OIL COMPOSITION FOR SHAVING

(71) Applicant: Christopher Lee Woodson, Eastvale, CA (US)

(72) Inventor: Christopher Lee Woodson, Eastvale, CA (US)

(21) Appl. No.: 17/173,971

(22) Filed: Feb. 11, 2021

Related U.S. Application Data
(63) Continuation-in-part of application No. 17/011,957, filed on Sep. 3, 2020.
(60) Provisional application No. 62/896,706, filed on Sep. 6, 2019.

(51) Int. Cl. A61K 8/9794; A61K 8/9789; A61Q 9/02; A61K 8/31; A61K 8/36; A61K 8/49

(52) U.S. Cl. CPC A61K 8/9794; A61K 8/9789; A61K 8/498; A61K 8/31; A61K 8/361; A61Q 9/02

(57) ABSTRACT

A therapeutic oil composition that includes a first oil phase, a second oil phase, a third oil phase, and a fourth oil phase. The first oil phase includes a coconut oil. The second oil phase includes a seed oil. The third oil phase includes a berry seed oil. The fourth oil phase includes a petroleum distillate. The coconut oil includes methyl salicylate. The coconut oil includes a saturated fatty acid in an amount of at least 85% by weight. The saturated fatty acid includes lauric acid in an amount of about 50% by weight. The seed oil includes linoleic acid in an amount of at least 60% by weight. The berry seed oil includes oleic acid in an amount of at least 1-11.7% by weight, linoleic acid in an amount of at least 52-66% by weight, and alpha-linolenic acid in an amount of at least 29-33% by weight.

### CROSS-REFERENCE TO RELATED APPLICATIONS

The current application is a continuation-in-part (CIP) application of a U.S. non-provisional application Ser. No. 17/011,957 filed on Sep. 3, 2020. The U.S. non-provisional application Ser. No. 17/011,957 claims a priority to U.S. Provisional Application No. 62/896,706 filed Sep. 6, 2019, both of which are incorporated by reference herein in their entirety.

### FIELD OF THE INVENTION

The present invention relates generally to shaving preparations and specifically to therapeutic oil compositions for shaving.

### BACKGROUND OF THE INVENTION

Razor and blade shaving preparations are provided in the art to lubricate the skin to allow the blade to glide easily over the skin and minimize irritation. These preparations also soften the hair and hold the hairs erect to facilitate cutting. Shaving preparations typically incorporate a fatty acid soap, a gelling and/or foaming agent, a lubricating agent, appropriate surfactants and other additives such as humectants, oils, preservatives, colorants and fragrances. Other formulations include “shaving assisting compositions” incorporating high amounts of emollient oils, silicones or alcoholic lotions. These compositions may be used as a prelubricant in conjunction with a conventional soap shaving formulation or may be used alone and applied directly to the skin or the shaving blade.

Although a number of products are currently available which cater to the needs of those with sensitive skin, better formulations are highly sought after for improving the lubricity of such compositions and providing adequate moisturization of the skin.

### BRIEF DESCRIPTION OF THE DRAWINGS

Some of the embodiments will be described in detail, with reference to the following figures, wherein like designations denote like members, wherein:

FIG. 1 depicts a method for preparing an oil composition according to some embodiments.

FIG. 2 depicts processes of a method for shaving using a therapeutic oil composition according to some embodiments.

FIG. 3 depicts processes of a method for shaving using a therapeutic oil composition according to other embodiments.

Unless otherwise specifically noted, articles depicted in the drawings are not necessarily drawn to scale.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below. Unless otherwise indicated, the drawings are intended to be read together with the specification and are to be considered a portion of the entire written description of this invention.

Razor and blade shaving preparations are provided in the art to lubricate the skin to allow the blade to glide easily over the skin and minimize irritation. These preparations also soften the hair and hold the hairs erect to facilitate cutting. Shaving preparations typically incorporate a fatty acid soap, a gelling and/or foaming agent, a lubricating agent, appropriate surfactants and other additives such as humectants, oils, preservatives, colorants and fragrances. Other formulations include “shaving assisting compositions” incorporating high amounts of emollient oils, silicones or alcoholic lotions. These compositions may be used as a prelubricant in conjunction with a conventional soap shaving formulation or may be used alone and applied directly to the skin or the shaving blade.

Although a number of products are currently available which cater to the needs of those with sensitive skin, better formulations are highly sought after for improving the lubricity of such compositions and providing adequate moisturization of the skin. The instant disclosure seeks to provide a therapeutic oil composition that addresses pseudofolliculitis barbae and skin irritation that develops from shaving and subsequent ingrown hairs.

The instant application seeks to provide therapeutic oil composition that can be applied to the skin and hair follicles for treating pseudofolliculitis barbae prior to shaving to prevent razor bumps before it starts forming. The therapeutic oil composition can be applied subsequent to shaving to sooth the skin and keep it from becoming irritated. Unlike prior solutions known in the art, the therapeutic oil composition has a formulation that: (1) uses oil compounds that remain on the epidermis and provides lubrication to the skin during the shaving process and thereby help prevent hair follicles from curling into the skin; and (2) uses oil compounds that absorb into the skin to relax it and reduce inflammation or a foreign body reaction due to shaving, which is the root cause of razor bumps.

The therapeutic oil composition includes a first oil phase, second oil phase, third oil phase, and fourth oil phase. The first oil phase includes a coconut oil. The coconut oil preferably includes saturated fatty acids in an amount of at least 85% by weight. The saturated fatty acid can include lauric acid in an amount of about 50% by weight. The coconut oil comprises monoi oil, which softens the skin and frustrates the ability of hair follicles to puncture the skin and thereby reduces the number of in-grown hairs. Not to be limited by theory, monoi oil is an infused tropical oil made from soaking the petals of Tahitian gardenias (also known as Tiaré flowers) in coconut oil and has antioxidant properties that when applied to skin is known to: reduce inflammation; moisturize the skin; and repair damaged skin.

Lauric acid is a medium-chain fatty-acid that possesses antimicrobial properties. The second oil phase includes a seed oil. The seed oil preferably includes linoleic acid in an amount of at least 60% by weight. Linoleic acid is an omega-6 fatty acid that regulates to inflammation. The seed oil can include safflower oil, another seed oil(s), or a combination of the two. Not to be limited by theory, safflower oil is extracted from the seeds of its plant which contains linoleic acid, that when applied to skin is known to: unclog pores; reduce ‘blackheads’; and stimulate the regeneration of new skin cells that help clear up scars and other blemishes from the surface of the skin. The third oil phase includes a berry seed oil. The berry seed oil preferably includes oleic acid, linoleic acid, and alpha-linoleic acid. Oleic acid is a monosaturated omega-9 fatty acid then when mixed with other fatty acids, such as linoleic acid, exhibits anti-inflammatory properties. The berry seed oil preferably includes oleic acid in an amount of 1-11.7% by weight, linoleic acid in an amount of at least 52-66% by weight, and alpha-linolenic acid in an amount of at least at 29-33% by weight. Alpha-linolenic acid is an omega-3 fatty acid that exhibits anti-inflammatory properties and is known for addressing skin diseases, such as psoriasis and eczema.

The berry seed oil preferably includes tocopherols, carotenoids, ellagic acid, or a combination of two or more thereof. Tocopherols are a class of organic chemical compounds that exhibit vitamin E (e.g., antioxidant) activity. The berry seed oil preferably includes raspberry seed oil, another seed oil(s), or a combination of the two. Raspberry seed oil is extracted from raspberry seeds and contains a high content of alpha-linolenic acid (the highest in any fruit seed oil) along with a high content of antioxidants and vitamin E. When applied to skin makes, the seed oil: acts as a UV protectant against a broad spectrum of UV radiation (e.g., UVA, UVB, UVC, or a combination of two or more thereof); reduced the destruction of collagen and inflammatory response; and prevent oxidative damage, which can lead to premature skin aging and skin cancer.

The fourth oil phase includes a petroleum distillate that preferably has a density of about 0.8-0.087 g/cm³. The petroleum distillate is preferably mineral oil. The mineral oil is used to reduce the brittleness of hair follicles and does not clog pores. The coconut oil comprises methyl salicylate, which acts as a rubefacient and analgesic (i.e. a skin-soothing agent). Mineral oil is a natural distillation product of petroleum with occlusive properties that when applied to skin is known to act as a shield to prevent external contaminants from penetrating the skin; and help retain ambient skin moisture.

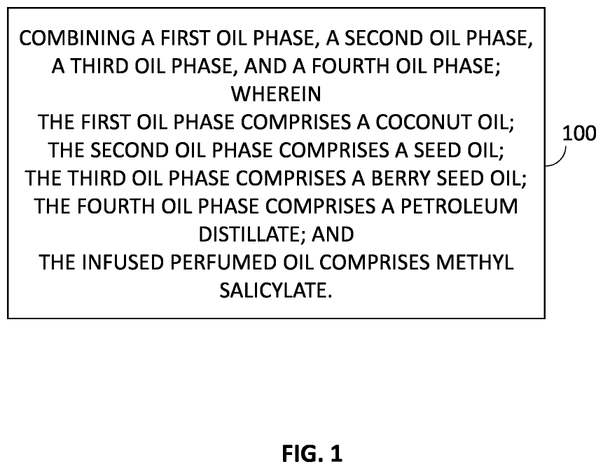

As reflected in FIG. 1, the therapeutic oil composition is preferably formed by combining the first oil phase, the second oil phase, the third oil phase, and the fourth oil phase. Here, the first oil phase includes coconut oil, the second oil phase includes a seed oil, the third oil phase comprises a berry seed oil, the fourth oil phase comprises a petroleum distillate, and the coconut oil includes methyl salicylate. In certain embodiments, the therapeutic oil composition includes: a first oil phase in an amount of 5% by weight; a second oil phase in an amount of 45% by weight; a third oil phase in an amount of 20% by weight; and a fourth oil phase in an amount of 30% by weight.

Here, the first oil phase includes a coconut oil, the second oil phase includes a seed oil, the third oil phase includes a berry seed oil, and the fourth oil phase includes a petroleum distillate. The coconut oil includes methyl salicylate, and a saturated fatty acid in an amount of at least 85% by weight. The saturated fatty acid includes lauric acid in an amount of about 50% by weight. The seed oil includes linoleic acid in an amount of at least 60% by weight. The berry seed oil includes oleic acid in an amount of at least 1-11.7% by weight, linoleic acid in an amount of at least 52-66% by weight, and alpha-linolenic acid in an amount of at least 29-33% by weight.

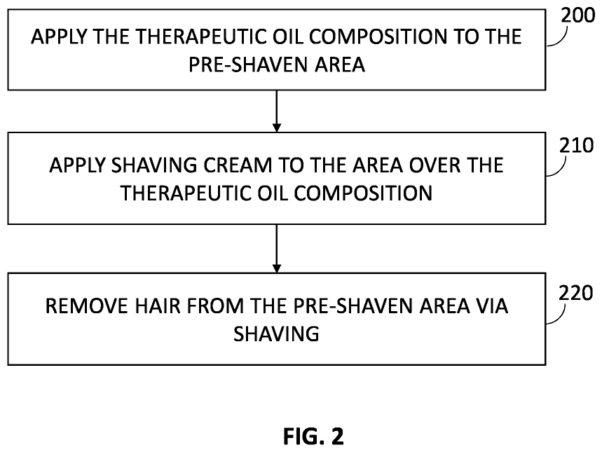

According preferred embodiments, the therapeutic oil composition is preferably used as a shaving balm. FIG. 2 depicts processes of a method for shaving using a therapeutic oil composition according to some embodiments. To begin, at Step 200 the therapeutic oil composition is applied to the pre-shaven area (i.e. the area where hair removal is desired). The pre-shaven area can be any place on a human or other mammal where hair removal is desired (e.g., face, head, legs, arms, back, etc.). The therapeutic oil composition is applied to the skin and hair follicles to address pseudofolliculitis barbae prior to hair removal to reduce the formation of razor bumps. Even more, antioxidants and other properties found in the therapeutic oil composition also treat the existing razor bumps as well as prevent new ones. At Step 210, shaving cream is applied to the pre-shaven area over the therapeutic oil composition. At Step 220, hair is removed from the pre-shaven area via shaving (i.e. the removal of unwanted hair from the mammalian epidermis with a razor).

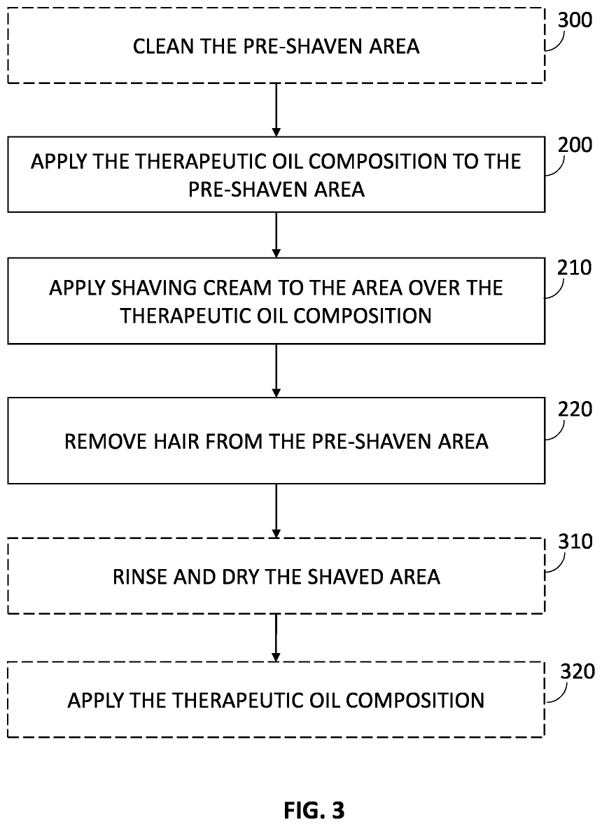

FIG. 3 depicts processes of a method for shaving using a therapeutic oil composition according to other embodiments. Specifically, FIG. 3 depicts a method that incorporates the method of FIG. 2. At Step 300, the pre-shaven area is cleaned (e.g., using soap and water). Subsequently, Steps 200, 210, and 220 are performed. At Step 310, the shaved area is rinsed and dried. At Step 320, the therapeutic oil composition is applied.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

Although the invention has been explained in relation to its preferred embodiment, it is to be understood that many other possible modifications and variations can be made without departing from the spirit and scope of the invention.

1. A therapeutic oil composition, comprising:
a first oil phase;
a second oil phase;
a third oil phase;
a fourth oil phase;
wherein
the first oil phase comprises a coconut oil;
the second oil phase comprises a seed oil;
the third oil phase comprises a berry seed oil;
the fourth oil phase comprises a petroleum distillate; and
the coconut oil comprises methyl salicylate.

2. The therapeutic oil composition of claim 1, wherein
the coconut oil comprises a saturated fatty acid in an amount of at least 85% by weight; and
the saturated fatty acid comprises lauric acid in an amount of about 50% by weight.

3. The therapeutic oil composition of claim 2, wherein the seed oil comprises linoleic acid in an amount of at least 60% by weight.

4. The therapeutic oil composition of claim 3, wherein the berry seed oil comprises:
oleic acid in an amount of at least 1-11.7% by weight;
linoleic acid in an amount of at least 52-66% by weight; and
alpha-linolenic acid in an amount of at least 29-33% by weight.

5. The therapeutic oil composition of claim 4, wherein the berry seed oil comprises tocopherols and carotenoids.

6. The therapeutic oil composition of claim 5, wherein the berry seed oil comprises ellagic acid.

7. The therapeutic oil composition of claim 6, wherein the petroleum distillate comprises a density of about 0.8-0.087 g/cm³.

8. The therapeutic oil composition of claim 7, wherein the coconut oil comprises monoi oil.

9. The therapeutic oil composition of claim 8, wherein the seed oil comprises safflower oil.

10. The therapeutic oil composition of claim 9, wherein the berry seed oil comprises raspberry seed oil.

11. The therapeutic oil composition of claim 10, wherein petroleum distillate is mineral oil.
